(19) United States
(12) Patent Application Publication
(10) Pub. No.: US 2002/0068361 A1
(43) Pub. Date: Jun. 6, 2002

(54) METHODS OF EVALUATING SPECIFIC CELLULAR FUNCTIONS OF RECEPTOR PROTEIN TYROSINE KINASES IN A LIGAND INDEPENDENT MANNER

(76) Inventor: DOUGLAS CLARY, SAN FRANCISCO, CA (US)

Correspondence Address: FOLEY & LARDNER, 3000K STREET NW, SUITE 500, WASHINGTON, DC, 20007, US

(21) Appl. No.: 09/057,150

(22) Filed: Apr. 7, 1998

Related U.S. Application Data
(60) Provisional application No. 60/043,207, filed on Apr. 8, 1997.

(51) Int. Cl. G01N 33/53; G01N 33/567; C12N 15/63; C12N 15/85; C12N 15/87

(52) U.S. Cl. 435/455; 435/7.1

(57) ABSTRACT

The invention relates to methods of evaluating the specific function of a receptor protein tyrosine kinase in cells. The methods activate the receptor in a ligand independent fashion. In addition, the invention includes methods of identifying compounds that modulate receptor protein tyrosine kinase function.

### RELATED APPLICATIONS

This application claims priority to U.S. Patent Application Serial No. 60/043,207, filed Apr. 8, 1997, entitled “Methods of Evaluating Specific Cellular Functions of Receptor Protein Tyrosine Kinases in a Ligand Independent Manner” by Douglas Clary (Lyon & Lyon Docket No. 222/299), which is hereby incorporated by reference herein in its entirety, including any drawings or figures.

### FIELD OF THE INVENTION

The invention described herein relates generally to methods of determining receptor protein tyrosine kinase function in cells or tissues. The methods activate a receptor in a ligand independent manner.

### BACKGROUND OF THE INVENTION

The following description of the background of the invention is provided to aid in understanding the invention, but is not admitted to be or describe prior art to the invention.

Cellular signal transduction is a fundamental mechanism whereby extracellular stimuli are relayed to the interior of cells. One of the key biochemical mechanisms of signal transduction involves the reversible phosphorylation of proteins. Phosphorylation of amino acids regulates the activity of mature proteins by altering their structure and function.

Phosphate most often resides on the hydroxyl moiety of serine, threonine, or tyrosine amino acids in proteins. Enzymes that mediate phosphorylation of cellular effectors fall into two classes. While protein phosphatases hydrolyze phosphate moieties from phosphoryl protein substrates, protein kinases transfer a phosphate moiety from adenosine triphosphate to protein substrates. The converse functions of protein kinases and protein phosphatases balance and regulate the flow of signals in signal transduction processes.

Protein kinases are divided further into two groups: receptor and non-receptor type proteins. Receptor protein tyrosine kinases comprise an extracellular region, a transmembrane region, and an intracellular region. Part of the intracellular regions of receptor protein kinases harbor a catalytic domain.

Receptor protein kinases are also divided into three classes based upon the amino acids they act upon. Some phosphorylate serine or threonine only, some phosphorylate tyrosine only, and some phosphorylate serine, threonine, and tyrosine. Receptor protein tyrosine kinases (RPTKs) are typically activated in the cell when a ligand binds to the extracellular region of the receptor. A model for ligand mediated activation of RPTKs features the ligand bringing the receptors within close proximity to one another. Some ligands are dimers and thereby bring the receptors that bind them into close proximity with one another. By bringing two RPTKs together, ligands place RPTK intracellular catalytic regions in close proximity with one another such that they cross-phosphorylate. Cross phosphorylation requires not only the dimerization process but also the occurrence of a conformational change preceding phosphorylation. The necessity of the conformational change preceding phosphorylation is illustrated by the fact that some RPTKs, such as the insulin receptor, are pre-dimerized and inactive before binding their activating ligands.

The presence of phosphate moieties on the RPTK intracellular regions constitutes a cellular signal that causes other signal transduction molecules to bind to the RPTK. In this manner, RPTKs propagate the extracellular signal to the cell nucleus thereby generating messages encoding proteins that cause cellular effects. Similar signaling cascades may be involved in, for example, cellular motility. This type of signal transduction pathway is described for the glial-derived growth factor (GDNF) activation of C-RET, an RPTK which phosphorylates tyrosine residues in target proteins. Jing et al., 1996, Cell 85:1113-1124; Trupp et al., 1996, Nature 381:785-789; Durbec et al., 1996, Nature 381:789-793.

Because RPTKs control a variety of cellular functions, any alteration in the normal function of an RPTK can result in an abnormal condition in an organism. Scientists are therefore aggressively searching for novel therapeutics for diseases that involve RPTKs.

A hurdle to designing therapeutics for RPTK-related diseases is identifying the function of a particular receptor and relating its activity to a disease or condition. One difficulty in identifying receptor function is that the ligands that activate the receptors are often unknown. In addition, even if a ligand is known, ligands can sometimes bind more than a single type of receptor and thus cause pleiotropic effects in an organism. Because alterations in the function of one RPTK can lead to a diseased state in an organism, determining the function of individual receptors is important for designing compounds that will prevent or treat these diseases.

RPTKs have been activated using antibodies with specific binding affinity to the extracellular region of one type of receptor. Spaargaren et al., 1991, J. Biol. Chem. 266: 1733-1739; Xiong et al., 1992, Proc. Natl. Acad. Sci USA 89; Clary et al., 1994, Mol. Biol. Cell. 5: 549-563. Because an antibody is bifunctional (it contains two polypeptide binding regions), an antibody can activate RPTKs by bringing them in close proximity with one another and thereby stimulating cross phosphorylation.

RPTKs have also been activated by fusing their intracellular regions to other polypeptides. These fused polypeptide constructs are termed “chimeras” since they are single polypeptides constructed from multiple regions of different proteins. Fazioli et al., 1991, Mol. Cell. Biol. 11: 2040-2048; Seedorf et al., 1992, Mol. Cell. Biol. 12: 4347-4356. One such RPTK chimera comprises an intracellular region of a RPTK fused to an epidermal growth factor (EGF) receptor extracellular region, thus activatable with the EGF ligand.

RPTK intracellular regions have also been activated in a ligand independent fashion by fusing intracellular polypeptides to them that bind bifunctional organic compounds. Spencer et al., 1993, Science 262: 1019-1024.

However, these ligand independent methods of activating RPTK intracellular regions are of restricted application. Simple antibody activation of RPTKs lacks versatility since a unique monoclonal antibody must be produced for each unique RPTK under study. The production of effective monoclonal antibodies for multiple RPTKs is prohibitive as monoclonal antibody production is both time consuming and inefficient.

Furthermore, even though the activation of chimeric constructs of RPTK intracellular regions confers versatility (the intracellular regions of RPTKs can be readily interchanged using recombinant DNA techniques), the ligands and organic compounds used to activate the chimeras can cause changes in cells or tissues other than those caused by the activated chimera. For example, the EGF ligand used to stimulate chimeric constructs containing the EGFR extracellular region can also activate endogenous EGFR expressed on the cell surface, sometimes existing at levels undetectable using currently known methods, and thereby interfere with the signal of the RPTK under study.

Thus, there still remains a need in the art for methods of activating RPTKs in ligand independent fashions that feature both specificity and versatility.

### SUMMARY

The invention relates to methods of evaluating the specific function of a receptor protein tyrosine kinase (RPTK) in cells or tissues. The methods activate a receptor in a ligand independent fashion. The methods of the invention overcome at least two problems associated with studying RPTKs and identifying compounds that modulate RPTK function. These problems are (1) lack of knowledge and availability of the ligand that activates an RPTK, and (2) ligands may activate more than one type of RPTK or activate undetectable levels of endogenous receptor, and thereby cause effects in a test cell that are not truly associated with the receptor under study.

Thus, in a first aspect, the invention features a method of evaluating RPTK function in cells. The method comprises the following steps: (a) transfecting a nucleic acid vector into cells, where the nucleic acid vector encodes a chimera comprising an extracellular region and an intracellular region, where the intracellular region is from the receptor protein tyrosine kinase; (b) contacting the cells with an antibody, where the antibody has specific binding affinity to the extracellular region; and (c) monitoring an effect on the cells to evaluate the function of the receptor protein tyrosine kinase.

The term “RPTK” refers to receptor protein tyrosine kinase. The invention includes any RPTK with an extracellular region that binds a ligand, a transmembrane region, and an intracellular region. RPTKs phosphorylate proteins on tyrosine residues. The nucleic acid sequence encoding an RPTK of the invention can be isolated from eukaryotic organisms.

The term “extracellular region” refers to a polypeptide portion of a RPTK of the invention that exists outside the cell membrane. A requirement of the extracellular region of chimeras of the invention is that it has specific binding affinity to another molecule, preferably an antibody of the invention. The extracellular region of the chimeras must not bind to any other protein on the cell surface and not normally be expressed on the surface of the specific cell type used in the invention.

The term “antibody” refers to an antibody (e.g., a monoclonal or polyclonal antibody), or antibody fragment, having specific binding affinity to RPTK's or fragments thereof, preferably the extracellular region of the RPTK's.

By “specific binding affinity” is meant that the antibody binds to target (RPTK) polypeptides with greater affinity than it binds to other polypeptides under specified conditions. Antibodies having specific binding affinity to a RPTK may be used in methods for detecting the presence and/or amount of a protein RPTK in a sample by contacting the sample with the antibody under conditions such that an immunocomplex forms and detecting the presence and/or amount of the antibody conjugated to the RPTK. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor.

The term “polyclonal” refers to antibodies that are heterogenous populations of antibody molecules derived from the sera of animals immunized with an antigen or an antigenic functional derivative thereof, preferably an extracellular region of a RPTK. For the production of polyclonal antibodies, various host animals may be immunized by injection with the antigen. Various adjuvants may be used to increase the immunological response, depending on the host species. “Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art. See, for example, Kohler, et al., Nature 256:495-497 (1975), and U.S. Pat. No. 4,376,110.

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target.

The term “ligand” as used herein refers to a polypeptide molecule which binds to the extracellular region of a receptor. Examples of ligands include, but are not limited to, nerve growth factor (NGF), which binds to the extracellular region of the TRK receptor, and GDNF which binds to a complex of C-RET and GDNFR-α.

The term “transmembrane region” as used herein refers to the region of a polypeptide that connects the extracellular region to the intracellular region, preferably in a chimera of the invention. The transmembrane region can be isolated from any transmembrane protein and is preferably from the same polypeptide source as the intracellular region. Transmembrane regions are often rich in hydrophobic amino acids, such as phenylalanine, tyrosine, tryptophan, valine, leucine, and isoleucine, which interact with fatty acid moieties of the membrane lipids and thereby anchor the chimera to the cell membrane.

The term “intracellular region” of an RPTK defines any region existing on the inner side of the cell. An intracellular region of a protein of the invention preferably includes the region of an RPTK with catalytic activity, and more preferably no other region of the RPTK.

DNA recombinant techniques known in the art provide the means of connecting an RPTK intracellular region to the extracellular region of another RPTK via a transmembrane region. In addition, the intracellular region of an RPTK can be attached to the extracellular region of a protein other than an RPTK, such as a receptor protein phosphatase.

The term “organism” relates to any living being comprised of at least one cell. An organism can be as simple as one eukaryotic cell or as complex as a mammal.

The term “eukaryote” refers to an organism comprised of cells that contain a nucleus. Eukaryotes are differentiated from “prokaryotes” which do not house their genomic DNA inside a nucleus. Prokaryotes include unicellular organisms such as bacteria while eukaryotes are represented by, for example, yeast, invertebrates, and vertebrates.

The term “mammal” refers preferably to such organisms as mice, rats, rabbits, guinea pigs, sheep, and goats, more preferably to cats, dogs, monkeys, and apes, and most preferably to humans.

The term “evaluating” describes the process of determining the cellular effect of activating an RPTK intracellular region in cells.

The term “function” refers to the effect of activating the intracellular region of one type of RPTK. The function of the intracellular region of one particular type of RPTK can be manifested in a change in the outward appearance of cells, or in a change in the interactions with proteins that bind to the intracellular region of the RPTK, and the like.

The term “transfecting” defines a number of methods to insert a nucleic acid vector or other nucleic acid molecules into a cellular organism. These methods involve a variety of techniques, such as treating the cells with high concentrations of salt, an electric field, detergent, or DMSO to render the outer membrane or wall of the cells permeable to nucleic acid molecules of interest or use of various viral transduction strategies.

The term “nucleic acid vector” relates to a single or double stranded circular nucleic acid molecule that can be transfected into cells and replicated within or independently of a cell genome. A circular double stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a chimeric receptor can be inserted into a vector by cutting the vector with restriction enzymes and ligating the two pieces together.

The term “contacting” as used herein refers to mixing a solution comprising one or more test compounds with a liquid medium bathing the cells of the methods. The solution comprising the one or more test compounds may also comprise another component, such as dimethylsulfoxide (DMSO), which facilitates the uptake of the one or more test compounds into the cells of the methods. The solution comprising the test compounds may be added to the medium bathing the cells by utilizing a delivery apparatus, such as a pipet-based device or syringe-based device. “Test compound” includes, but is not limited to, both antibodies and compounds of the invention.

The term “compound” refers to a peptide of less than twenty, preferably less than fifteen, preferably less than ten, or most preferably less than eight amino acids in length. The term “compound” preferably refers to a peptidomimetic, more preferably refers to a non-peptide organic molecule, and most preferably refers to a nonpeptide synthetic organic molecule. Examples of compounds are included in the Description of the Invention, herein.

The term “peptidomimetic” refers to an organic molecule with a structure related to a natural peptide, and which can exhibit or block the biological activities of the natural peptide.

The term “monitoring” refers to observing the effect of adding test compounds to the cells. The effect can be manifested in cell phenotype or in the interaction between an RPTK intracellular region and a natural binding partner.

The term “effect” preferably describes a change or an absence of a change in cell phenotype. “Effect” can also describe a change or an absence of a change in the catalytic activity of the RPTK. “Effect” can also describe a change or an absence of a change in an interaction between the RPTK and a natural binding partner.

The term “cell phenotype” refers to the outward appearance of a cell or tissue or the function of the cell or tissue. Examples of cell phenotype include, but are not limited to, cell size (reduction or enlargement), cell shape, cell proliferation (increased or decreased numbers of cells), cell differentiation (changes in physiological state, cell survival, apoptosis (cell death), or the utilization of a metabolic nutrient (e.g., glucose uptake). Changes or the absence of changes in cell phenotype are readily measured by techniques known in the art.

The term “cell proliferation” refers to the rate at which a group of cells divides. The number of cells growing in a vessel can be quantified by a person skilled in the art when that person visually counts the number of cells in a defined area using a common light microscope. Alternatively, cell proliferation rates can be quantified by laboratory apparatae that optically measure the density of cells in an appropriate medium.

Cell differentiation and survival are phenomena simply measured by methods in the art. These methods can involve observing the number of cells or the appearance of cells under a microscope with respect to time (days).

The term “apoptosis” as used herein refers to programmed cell death. It has been shown by example herein that activation of C-RET can prolong cell survival and decrease the probability of cell death.

The term “catalytic activity”, in the context of the invention, defines the rate at which an RPTK intracellular region phosphorylates a substrate. Catalytic activity can be measured, for example, by determining the amount of a substrate converted to a phosphorylated product as a function of time. Catalytic activity can be measured by methods of the invention by holding time constant and determining the concentration of a phosphorylated substrate after a fixed period of time. Phosphorylation of a substrate occurs at the active-site of a protein kinase. The active-site is normally a cavity in which the substrate binds to the protein kinase and is phosphorylated.

The term “interaction”, in the context of the invention, describes a complex formed between a RPTK and a natural binding partner or compound, preferably between a RPTK intracellular region and a natural binding partner or compound.

The term “complex” refers to an assembly of at least two molecules bound to one another. Signal transduction complexes often contain at least two protein molecules bound to one another. For instance, a protein tyrosine receptor protein kinase, GRB2, SOS, RAF, and RAS assemble to form a signal transduction complex in response to a mitogenic ligand.

The term “natural binding partner” refers to polypeptides that bind to a RPTK. Natural binding partners can play a role in propagating a signal in a protein kinase signal transduction process. A change in the interaction between a RPTK and a natural binding partner can manifest itself as an increased or decreased probability that the interaction forms, or an increased or decreased concentration of the RPTK/natural binding partner complex.

A RPTK natural binding partner can bind to a RPTK intracellular region with high affinity. High affinity represents an equilibrium binding constant on the order of 10⁻⁶ M or less. In addition, a natural binding partner can also transiently interact with a RPTK intracellular region and chemically modify it. Protein kinase natural binding partners are chosen from a group consisting of, but not limited to, SRC homology 2 (SH2) or 3 (SH3) domains, other phosphoryl tyrosine binding (PTB) domains, guanine nucleotide exchange factors, protein phosphatases, and other protein kinases. Methods of determining changes in interactions between protein kinases and their natural binding partners are readily available in the art.

In preferred embodiments of the invention, the method of evaluating a function of a RPTK involves transfecting nucleic acid vectors that encode chimeras that contain an extracellular region of a RPTK selected from the group consisting of TRK, EGFR, PDGFR, and RET and intracellular regions of an orphan RPTK. Preferably, the orphan RPTK is selected from the group consisting of C-RET, SEK, MCK-10, AXL, TYRO3, MER, EPH, ECK, EEK, ERK, ELK, EHK1, EHK2, SEK, HEK, HEK2, MYK1, CEK9, MYK2, MDK1, IRR, CCK4, RYK, DDR, TYRO10, ROS, LTK, ALK, ROR1, ROR2, and TOR. Most preferably, the orphan RPTK is C-RET.

The term “orphan RPTK” as used herein refers to any receptor whose primary amino acid sequence is closely related to the primary amino acid sequence of the RPTK family. It is called an orphan receptor because no ligand has been identified which directly activates it. In addition, the term “orphan receptor” as used herein refers to an RPTK without a known function. Alternatively, orphan RPTKs may be activated by a ligand known in the art, but the ligand has not yet been shown to directly activate the receptor of interest in the art.

The nucleic acid sequences of known RPTKs, and thereby their amino acid sequences, can be readily determined by searching a sequence database with the three or four letter name of the RPTK. Examples of sequence databases are the EMBL and Genbank databases. In addition, the sequence database can be searched by the database accession number for an RPTK. Furthermore, the databases can be searched using sequences of the RPTKs themselves.

Genbank database accession numbers are designated in parenthesis for the sequence of the following RPTK genes: axl (M76125); tyr03 (D17517); mer (U08023); eph (M18391); eck (M59371); eek (X59290); erk (D31661); elk (M59814); ehkl (S38024); ehk2 (S68030); sek (S57168); hek (M83941); hek2 (S65702); myk1 (U06834); cek9 (Z19060); and mdk1 (X79082). If desired, an RPTK gene can be cloned from a cDNA library with nucleic acid probes designed from the nucleic acid sequence provided in the database.

RPTKs can be identified by designations other than those disclosed herein. For example, the RPTK AXL is also referred to in the art as ARK and UFO. TYRO3 is also referred to as SKY, RSE, TIF, and BRT. MER can be referred to as TYRO12, EYK, and C-RYK. ECK is also referred to as SEK2. ERK is also referred to as HEK5, TYRO5, CEK5, and NUK. ELK can be referred to as CEK6 and HEK6. EHK1 has been referred to as HEK7, CEK7, and BSK. EHK2 is also referred to as HEK12. SEK can be referred to as HEK8, CEK8, TYRO1, and MPK3. HEK has been referred to as HEK4, CEK4, TYRO4, and MEK4. HEK2 is also referred to as CEK10 and TYRO6. MYK1 is also referred to as TYRO11, HPTK5, and HTK. CEK9 has been also referred to as HEK9. MDK2 is also referred to as MDK1-T1 or T2, EHK-3, and HEK11.

In addition, other as yet unidentified orphan RPTKs exist that can be identified by methods well-known to those skilled in the art. RPTKs, like protein kinases in general, share a 250-300 amino acid domain that can be subdivided into 12 distinct subdomains that comprise the common catalytic core structure. These conserved protein motifs can be (and have been) exploited using PCR-based cloning strategies well-known in the art to identify other RPTKs.

RPTK nucleic acid sequences can also be identified by: analyzing sequenced, but unidentified, DNA or RNA or polypeptide sequences from public databases; by probing DNA libraries (e.g., cDNA and genomic libraries) using degenerate probes to sequences conserved in various families and sub-families of such kinases; or by PCR-based cloning using degenerate primers based on conserved sequences for various families and sub-families. For the probing or PCR-based approaches, once one or more clones are identified having sequences corresponding to the probe or primers, the genomic or cDNA insert in that clone can be at least partially sequenced by routine methods to confirm that the cloned sequence actually corresponds to a RTPK, and to provide a unique sequence identifying the full gene. Possession of such a unique sequence provides the full gene sequence by following routine techniques. Likewise, a probe sequence based on a specific homologous RTPK sequence from a different mammalian species can be used to detect the corresponding human version of that gene.

In preferred embodiments of the method of evaluating a function of a RPTK, the cells of the invention and the extracellular region of the chimera are from different species. Preferably, the cells are mammalian, while the extracellular region of the RPTK is isolated from a chicken. The invention also features methods of evaluating a cellular function of an RPTK, in which the antibodies of the invention have specific binding affinity to a TRK extracellular region isolated from a chicken and where the effect is a change in cell phenotype.

In a second aspect, the invention features a method of identifying one or more compounds that modulate the function of a receptor protein tyrosine kinase in a cell. The method comprises the following steps: (a) transfecting a nucleic acid vector into a cell, where the nucleic acid vector encodes a chimera comprising an extracellular region and an intracellular region, where the intracellular region is from the receptor protein tyrosine kinase; (b) contacting the cells with one or more compounds; (c) contacting the cells with an antibody, where the antibody has specific binding affinity to the extracellular region and (d) monitoring the effect on the cell to identify compounds that modulates the function of the receptor protein tyrosine kinase.

The term “modulates” refers to the ability of a test compound (or “modulator”) to alter the function of a RPTK. A modulator preferably activates or inhibits the activity of a RPTK depending on the concentration of the compound exposed to a RPTK. A modulator can enhance or inhibit the catalytic activity of the RPTK by binding to the RPTK. By binding to the RPTK, the compound may inhibit the catalytic activity of the RPTK by blocking interactions between the RPTK and a substrate that it phosphorylates. A compound may activate the RPTK by bringing RPTKs into close proximity with one another such that they cross phosphorylate and thereby activate more effectively, or by increasing the probability that a catalysis-dependent conformational change occurs in the RPTK.

In addition, a modulator can inhibit the interaction between a RPTK and a natural binding partner by blocking interactions between amino acids at the interface of the complex. In addition, a modulator may inhibit the activity of a natural binding partner which acts upon the RPTK. Alternatively, a modulator can enhance the interaction between a RPTK and a natural binding partner by forming additional favorable interactions between the two molecules at the complex interface. A modulator preferably inhibits the catalytic activity of a protein kinase, more preferably activates or inhibits the catalytic activity of a protein kinase depending on the concentration of the compound exposed to the protein kinase, or most preferably activates the catalytic activity of a protein kinase.

The term “activates” refers to increasing the cellular function of a RPTK. The RPTK function is preferably the interaction with a natural binding partner or catalytic activity. An activator of C-RET is a cellular factor that is responsible for the cellular response of C-RET after it binds to GDNF. C-RET activators include, but are not limited to GRB2 and SOS. Compounds can enhance the interaction between C-RET and GRB2 or SOS and effectively activate the cellular function of C-RET.

The term “inhibit” refers to decreasing the cellular function of a RPTK. The RPTK function is preferably the interaction with a natural binding partner or catalytic activity. A hypothetical example of a protein that decreases the function of the RPTK, C-RET, is a protein phosphatase that dephosphorylates the activated receptor and thereby decreases C-RET's ability to recruit other proteins necessary for the C-RET activation response, such as GRB2 and SOS.

The term “signal transduction pathway” refers to the molecules that propagate an extracellular signal through the cell membrane to become an intracellular signal. This signal can then stimulate a cellular response. The polypeptide molecules involved in signal transduction processes are typically receptor and non-receptor protein tyrosine kinases, receptor and non-receptor protein phosphatases, SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), nucleotide exchange factors, and transcription factors.

The term “substrate” as used herein refers to a molecule phosphorylated by a RPTK. RPTKs phosphorylate substrates on tyrosine amino acids.

In preferred embodiments of methods of identifying compounds that modulate the function of an RPTK, the extracellular region of the chimera is an extracellular region of a RPTK selected from the group consisting of TRK, EGFR, PDGFR, and RET, and the intracellular region is an intracellular region of an orphan RPTK. Preferably the orphan RPTK is selected from the group consisting of C-RET, SEK, MCK-10, AXL, TYRO3, MER, EPH, ECK, EEK, ERK, ELK, EHK1, EHK2, SEK, HEK, HEK2, MYK1, CEK9, MYK2, MDK1, IRR, CCK4, RYK, DDR, TYRO10, ROS, LTK, ALK, ROR1, ROR2, and TOR. Most preferably, the orphan RPTK is C-RET.

In preferred embodiments of the method of identifying compounds that modulate a function of a RPTK, the cells of the invention and the extracellular region of the chimera are from different species. Preferably, the cells are mammalian, while the extracellular region of the RPTK is isolated from a chicken. The invention also features methods of evaluating a cellular function of an RPTK, in which the antibodies of the invention have specific binding affinity to a TRK extracellular region isolated from a chicken and where the effect is a change in cell phenotype. The change in cell phenotype includes, but is not limited to, a change in the catalytic activity of the RPTK intracellular region and a change in an interaction between the intracellular region and a natural binding partner.

In a third aspect, the invention relates to methods of identifying compounds that modulate the function of C-RET RPTK comprising the following steps: (a) transfecting C-RET into cells; (b) contacting the cells with one or more compounds; and (c) monitoring an effect on said cells. The effect includes, but is not limited to, a change or an absence of a change in a cellular event selected from the group consisting of: cell phenotype, which includes, but is not limited to, apoptosis and cell proliferation; catalytic activity of C-RET; and the interaction between C-RET and a natural binding partner.

The summary of the invention described above is not limiting and other features and advantages of the invention will be apparent from the following detailed description of the invention, and from the claims.

### DETAILED DESCRIPTION OF THE INVENTION

The invention relates to methods of evaluating the specific function of a receptor protein tyrosine kinase (RPTK) in cells or tissues by activating a receptor in a ligand-independent fashion.

The ligand-independent methods of evaluating the specific function of a RPTK overcome at least two problems associated with studying RPTKs and identifying compounds that modulate RPTK function. These problems are (1) lack of knowledge about and availability of the ligand that activates a RPTK, and (2) ligands may activate more than one type of RPTK or activate undetectable levels of endogenous receptor, and thereby cause effects in a test cell that are not truly associated with the receptor under study.

The invention features antibody-induced activation of chimeric RPTKs constructed from polypeptides of at least two different proteins. The chimeras contain the extracellular and transmembrane region from one type of RPTK and an intracellular region from another type of RPTK. This chimeric approach confers both specificity and versatility towards evaluating RPTK cellular function.

The methods of the invention incorporate a novel cross species feature with respect to the chimeric constructs and cells utilized in these methods. The cross species feature ensures that the only receptor appreciably activated in response to an antibody of the invention is the chimeric construct expressed on the surface of cells. This cross species feature represents an improvement over the existing techniques which can activate multiple receptors on cell surfaces.

Therefore, the methods set forth herein provide for test compound-induced activation of specific chimeric receptors expressed on the surface of cells under study. As an example, the extracellular region of the chimeric construct may originate from chickens while the cells in which the chimeric construct is expressed originate from rats. Examples set forth herein demonstrate that antibodies raised specifically to the chicken extracellular region of the chimeric construct do not cross-react with other proteins expressed on rat cell surfaces.

RPTKs are essential regulatory molecules controlling a variety of cellular functions. For this reason, any alteration in the function of a RPTK can result in an abnormal condition in an organism. Just two of the many functions controlled by RPTKs are cell proliferation and cell survival.

I. Cell Proliferation and Signal Transduction

Alterations in the function of an RPTK that normally regulates cell proliferation can lead to enhanced or decreased cell proliferative conditions evident in certain diseases. Aberrant cell proliferative conditions include cancers, fibrotic disorders, mesangial disorders, abnormal angiogenesis and vasculogenesis, poor wound healing, psoriasis, restenosis, and inflammation.

Fibrotic disorders relate to the abnormal formation of the cellular extracellular matrix. An example of a fibrotic disorder is hepatic cirrhosis. Hepatic cirrhosis is characterized by an increased concentration of extracellular matrix constituents resulting in the formation of a hepatic scar. Hepatic cirrhosis can cause diseases such as cirrhosis of the liver.

Mesangial cell proliferative disorders occur due to the abnormal proliferation of mesangial cells. Mesangial proliferative disorders include various human renal diseases, such as glomerulonephritis, diabetic nephropathy, malignant nephrosclerosis, thrombotic microangiopathy syndromes, transplant rejection, and glomerulopathies.

Angiogenic and vasculogenic disorders result from excess proliferation of blood vessels. Blood vessel proliferation is necessary in a variety of normal physiological processes such as embryonic development, corpus luteum formation, wound healing and organ regeneration. However, blood vessel proliferation is also essential in cancer tumor development. Other examples of blood vessel proliferative disorders include arthritis, where new capillary blood vessels invade the joint and destroy cartilage, and ocular diseases (such as diabetic retinopathy), where new capillaries in the retina invade the vitreous, bleed and cause blindness. Conversely, disorders related to the shrinkage, contraction or closing of blood vessels, such as restenosis, are also implicated in adverse regulation of RPTKs.

In addition to cell proliferation, some RPTKs regulate cell survival and cell death. The RPTK C-RET, for example, phosphorylates polypeptide substrates on tyrosine amino acids in neuronal cells. This RPTK is implicated in development and survival of enteric, sympathetic, and sensory neurons upon stimulation by the ligand glial derived neurotrophic factor (GDNF). Jing et al., 1996, Cell 85:1113-1124; Trupp et al., 1996, Nature 381:785-789; Durbec et al., 1996, Nature 381:789-793.

Lack of function mutations in c-ret can lead to Hirschsprung's disease, for example, which manifests itself as a decrease in intestinal tract innervation in patients. Thus, compounds that activate c-ret are potential therapeutic agents for the treatment of neurodegenerative disorders, including, but not limited to, Hirschsprung's disease, Parkinson's disease, Alzheimer's disease, and amyotrophic lateral sclerosis. Compounds that inhibit cret function are possible anti-cancer agents as overexpression of ret in cells is implicated in cancers, such as cancer of the thyroid.

II. Method of Determining the Function of RPTKs

A hurdle to designing therapeutics for RPTK-related diseases is identifying the specific function of a particular receptor. A reason for this hurdle is that the ligands that activate the RPTKs are often unknown. In addition, even if an ligand is discovered, many ligands can bind more than a single type of receptor and thus cause pleiotropic effects in an organism. Because alterations in the function of one type of RPTK can lead to the diseased states described herein, determining the function of individual receptors is important for designing compounds that diagnose or treat these diseases.

The invention addresses these specific hurdles to drug design by featuring methods that activate the intracellular regions of RPTKs in a ligand independent manner. The invention relates to methods of evaluating the specific function of an RPTK by specifically activating the receptor in a ligand independent fashion.

The invention features antibody-induced activation of chimeric polypeptide constructs comprising an RPTK intracellular region. This approach confers both specificity and versatility towards evaluating RPTK cellular function.

The chimeric polypeptide constructs confer a versatile and non-invasive approach to evaluating RPTK cellular function. The chimeras comprise a polypeptide extracellular region, a transmembrane region, and an intracellular region of an RPTK.

The chimeric feature of the invention provides versatility as the intracellular region of any known RPTK can be incorporated readily into a chimeric construct using DNA recombinant techniques existing in the art. In addition, the chimeric constructs utilized in the invention conserve receptor structure since the extracellular and transmembrane regions of one RPTK are preferably linked to the intracellular region of another RPTK. Because the structural organization of RPTKs are similar, connecting the extracellular regions and intracellular regions of different RPTKs should not significantly alter polypeptide structure. Thus, utilizing chimeric RPTK receptors confers versatility without significantly perturbing receptor structure.

The chimeric feature of the invention also confers specificity by utilizing antibody-induced activation of chimeras. The chimeras can be activated in a specific manner by an antibody with specific binding affinity to the polypeptide extracellular region of the chimera. The antibodies can then place the intracellular regions of the chimera in close proximity to one another and subsequently induce activation of the intracellular region in a specific fashion.

To achieve specific activation of chimeric receptor constructs, the invention features a cross species chimera/cell system. In this system, the extracellular region of the chimeric receptor is selected from an organism distantly related to the organism from which the cells expressing the chimeric receptor are derived. This cross species strategy ensures that endogenous receptors expressed on the cell surface are not activated by the antibodies raised against the extracellular region of the chimeric construct. For example, the antibodies of the invention have specific binding affinity for the TRK extracellular region from chicken. The antibodies having specific binding affinity for the chicken TRK extracellular region will consequently not cross react with the endogenous TRK receptor of rat or human cells. Thus, the antibodies of the invention prevent cross reactivity with endogenous receptors expressed on the surface of mammalian cells since the antibodies have specific binding affinity to a distinct receptor of a different type of organism. The antibody can be reacted with cells that are not transfected with the chimeric construct of the invention in control experiments testing the system.

Activation of chimeric receptors by antibodies is more specific than activation of chimeric receptors by ligands. Antibodies can specifically bind a definite type of receptor while it is possible for ligands to activate multiple receptors on the cell surface. Moreover, ligands can activate any endogenous receptor that may be expressed on the cell surface which will interfere with the signal followed from the chimeric receptor. Even undectable levels of endogenous receptor expressed on the cell surface can interfere with the signal followed in the methods of the invention.

In addition, activation of chimeric receptors by antibodies is more specific than activation of chimeric receptors by bifunctional organic molecules. Unlike many small organic molecules used to activate chimeric constructs, antibodies will not typically traverse the cell membrane or endocytose to the cell interior and bind to other cellular components. Even if the antibody does translocate to the cell interior, it will most likely not interact with cellular components and will therefore not elicit a cellular response. Thus, antibody-induced activation of chimeras, as opposed to ligand or organic molecule-induced activation of chimeras, specifically activates RPTK intracellular regions without complicating side effects.

III. Other Embodiments

Recombinant DNA techniques pertaining to the invention, nucleic acid vectors, the nucleic acid elements of these vectors, the types of cells or tissues that can harbor these vectors, methods of delivering these vectors to cells, methods of producing and purifying antibodies, methods of constructing hybridomas that produce these antibodies, methods of detecting signaling molecule complexes, methods of detecting interactions with natural binding partners, and methods of monitoring changes in cells are well known to persons skilled in the art. Descriptions of these techniques and methods are disclosed in detail with respect to the protein PYK-2 in PCT publication WO 96/18738. This publication is incorporated herein by reference in its entirety, including any drawings. Those skilled in the art will readily appreciate that such descriptions are applicable to the present invention and can be easily adapted to it.

Other methods associated with the invention are described in the examples disclosed herein.

### EXAMPLES

The examples below are not limiting and are merely representative of various aspects and features of the present invention. The examples demonstrate methods of specifically activating RPTKs in a ligand independent manner. The examples also demonstrate the specificity as well as the versatility of the methods.

### Example 1

### Specific Activation of RPTKs by Antibodies

The following demonstrate that the intracellular region of an RPTK could be activated when it was fused to a TRK extracellular region isolated from chicken. This chimeric construct was activated by an antibody that had specific binding affinity for the TRK extracellular region from chicken organisms. In particular, the chimeric constructs were activated by polyclonal antibody preparation raised against the chicken TRKA receptor. The antibody specifically activated the chimeric construct in cells since the cells did not express any other proteins harboring regions homologous to the TRK extracellular region from chicken. Thus, the antibodies distinguished between endogenous TRKA receptor expressed on the surface of the rat cells and the chimeric construct, containing the chicken TRKA extracellular region, also expressed on the surface of the cells.

The antibody preparation used in the experiments, called CTA, was a rabbit antibody raised against the entire extracellular domain of chicken TRKA expressed in COS cells. The antibody was specific for chick TRKA and did not cross-react with the rat receptor, nor did it elicit biological activities in rat cells. It did, however, strongly recognize the chick TRKA receptor by immunoblot or immunoprecipitation and acted as a ligand which activated the chicken receptor. A useful control for these experiments was to activate the endogenous rat TRKA receptor without influencing the introduced chimeric receptor. This specific activation was achieved through the use of the RTA IgG preparation, which recognized the rat receptor but not the chick receptor.

An assumption made in the experiments was that the chimeric receptor activated by the CTA antibody did not in turn activate the endogenous receptor. The assumption was tested by expressing both rat and chicken TRKA receptors in the same cell and activating them with either neurotrophic growth factor (NGF), which binds both receptors, or with the CTA antibody, which binds only the chick receptor. While the chick receptor became phosphorylated by both treatments, activation of the rat receptor was detected only after NGF addition. Thus, the activated chick receptor did not in turn activate the rat receptor. The effects of the CTA antibody preparation were therefore not attributable to activation of endogenous TRKA receptors.

The sympathetic and sensory neurons used in the experiments were cultured in a defined medium. Hawrot and Patterson, 1979, Methods Enzymol. 53: 574-584. Sympathetic neurons were isolated from superior cervical ganglia dissected from E20-E21 rat fetuses, while dorsal root ganglion sensory neurons were obtained from E16-E18 rats. The ganglia were treated with 0.25% trypsin for 10 minutes, washed, and triturated to obtain a single cell suspension. Sensory neurons were preplated for 1 hour on tissue culture plastic to deplete adherent cells. Sensory and sympathetic neurons were infected with adenoviruses encoding the chimeric RPTK proteins for two hours on collagen I-coated tissue culture plastic in the presence of NGF, and the cells were then washed and allowed to recover for two to four additional hours in the presence of NGF. After the recovery period, the cells were washed extensively to remove the growth factor, and plated onto polylysine-laminin coated chamber slides with 15 μg/mL RTA or CTA IgG, or no addition. After an additional two days (sensory) or three days (sympathetic), the cultures were stained with calcein AM (1 μg/mL) for 45 minutes, mounted and examined by immunofluorescence. Generally, five disperse fields representing 7% of the well were photographed and the number of surviving neurons quantitated. The conditions for immunoprecipitation and immunoblotting used in the experiments were described previously (Clary et al., 1994, Mol. Biol. Cell 5: 549-563) with modifications as follows. Immunoblots were performed in Tris-buffered saline containing 0.1% Triton X-100, and filter blocking and antibody incubations were performed in the same buffer with 1% BSA. In other experiments, anti-phosphotyrosine detection was performed with biotinylated-4G10 monoclonal antibody followed by a peroxidase avidin-biotin complex. Other antibodies were detected with peroxidase coupled anti-mouse IgG, anti-rabbit IgG, or protein A as indicated. All peroxidase conjugates were detected using a chemiluminescence protocol (Pierce Chemical Co. Rockford, Ill.).

### Example 2

### Construction of Adenoviral Vectors Expressing Receptor Constructs

In order to test the biochemical effect of receptor tyrosine kinases in chimeric receptor constructs, a method for efficient gene transfer was developed which would work with multiple cell lines as well as primary neuronal cultures. A recombinant adenoviral system was developed, as adenovirus exhibits a wide host range, and can infect and direct gene expression in post-mitotic neurons.

Recombinant adenoviruses were generated by in vivo ligation. The transfer vector (pAdRSVOES-) contained a 5′ adenovirus packaging sequences, the Rous Sarcoma Virus long terminal repeat promoter, a polylinker ending in the restriction site BstBI, the SV40 poly A region, 3′ adenoviral sequences (which could be used for recombination), and sequences for propagation in E. coli. The viral DNA used for generation of recombinant viruses was derived from a virus which expressed the β-galactosidase gene driven by the RSV promoter, and which contained a BstBI site between the β-galactosidase gene and the poly A region. This vector allowed screening for recombinant plaques based on the presence or absence of β-galactosidase activity, similar to a system described by Schaack and colleagues. Schaack et al., 1995, J. Virol. 69: 3920-3923. This virus lacks the E3 region.

The cells utilized for the generation of recombinant adenovirus were HEK293 cells. Early passage HEK293 cells (Graham et al., 1977, J. Gen. Virol 36: 59-72) were maintained in Dulbecco's modified Eagles medium+10% calf serum. Cells infected with recombinant adenovirus were detected as plaques in the cell monolayer. Typically, 5 μg of transfer vector plasmid DNA cleaved by digestion with BstBI were coprecipitated with 2 μg of viral DNA also cleaved with BstBI. HEK293 monolayers were transfected with the DNA and cultured from five to seven days to allow plaques to appear. The monolayers were then stained with 25 μg/mL X-GAL (5-bromo-4-chloro-3-indolyl-β-D-galactopyranoside) for several hours to identify non-recombinant (stained) plaques. Putative recombinant plaques were screened for expression of the transgene by infection of HEK293 cultures followed by immunohistochemistry. Viruses which were positive for transgene protein expression were picked and subjected to several rounds of plaque purification prior to amplification and purification on cesium chloride gradients. Banded viruses were diluted five-fold with dilution buffer (Curiel et al., 1991, Proc. Natl. Acad. Sci. USA 88: 8850-8854) and stored at −80° C. Approximate titers of the virus preparations were determined immunohistochemically on HEK293 cultures. Although recombinant viruses were generated through recombination into the viral genome or through cotransfection of cleaved DNAs, higher recovery of recombinants was achieved through the cleaved DNA method. The putative recombinant viruses were screened for expression of the kinase transgene by immuno-histochemistry.

Immuno-histochemistry techniques were performed on cultures of sympathetic neurons infected with the RSV KP or RSV c-ret viruses and fixed and stained as described. Lefcort et al., 1996, J. Neurosci. 16: 3704-3713. The primary antibodies were the CTA IgG, and mouse anti—tyrosine hydroxylase. The primaries were detected with Cy3-anti rabbit IgG and fluorescein-anti mouse IgG.

### Example 3

### Expression and Activation of C-RET, SEK, and MCK-10 Chimeras

Once the adenoviral expression vectors were transfected into the neuronal cells, an initial survey of RPTK expression was performed for these cells. Several uncharacterized kinases were expressed in these cell types. For example, the RPTK SEK was expressed in embryonic rat motor neurons, while MCK-10 (DDR) and C-RET were expressed in both motor and sympathetic neurons.

DNA constructs were generated, which encoded the transmembrane and cytoplasmic domains from human MCK-10, C-RET, mouse SEK, and rat TRKA fused to the chick TRKA extracellular domain. These chimeric receptor constructs were then inserted into the adenovirus transfer vector, and recombinant viruses were generated and purified. Rat C6 glioma cells were infected with the recombinant viruses, and the resulting chimeric receptors were analyzed via immunoblot analysis.

The chimeric RET protein appeared to be approximately 155 kDa as expected. The chimeric SEK protein was approximately 150 kDa, which is very similar to the chimeric and non-chimeric TRKA constructs, which have a molecular weight of approximately 140 kDa, and consistent with a fully processed, mature receptor. The MCK-10 chimeric protein appeared in three bands, of molecular weights of approximately 160, 120, and 105 kDa. The two lower molecular weight forms were quite abundant and probably represent immature forms of this chimera.

Once it was determined that the chimeric receptors were expressed in the cells, their activity was determined by monitoring the degree that they autophosphorylated after stimulation by ligand.

The ability to activate the chimeras was evaluated by incubating the infected C6 monolayers with 50 ng/mL NGF for five minutes. The receptors were immunoprecipitated with the CTA antibody, immunoblotted, and probed either with the anti-phosphotyrosine antibody 4G10 or with the CTA antibody preparation. NGF-dependent activation of the C-RET and SEK chimeras was detected by the anti-phosphotyrosine antibody. The chimeric C-RET receptor showed some activation in the absence of ligand addition. Phosphotyrosine was not detected for the MCK-10 chimeric protein, either before or after incubation with NGF.

The PC12 and C6 glioma cells used in the experiments were cultured as follows. PC12 cultures (Greene et al., 1987, Methods Enzymol. 147: 207-216) were maintained in RPMI medium containing 10% horse serum and 5% fetal calf serum. For differentiation experiments the medium was changed to RPMI containing IX N2 supplement and 0.1% BSA, and the cells were grown on a collagen I substrate. For PC12 cell survival, the cells were grown in RPMI containing 0.1% BSA. All cultures also contained IX penicillin/streptomycin. For adenoviral infections, PC12 cells were incubated overnight with recombinant viruses at a multiplicity of infection (MOI) between 1 and 10. The cells were then washed and replated either into differentiation or survival conditions, with either no addition or addition of 50 ng/mL (NGF) or 20 μg/mL CTA IgG for two days.

For differentiation experiments, the cell cultures were fixed with 2% paraformaldehyde and the percentage of cells bearing processes longer than 1 cell diameter was determined. For survival, the cultures were incubated with 0.05% MTT for 1.5 hours to stain living cells, and the relative number of cells surviving in each condition was determined.

C6 glioma cells were grown in Ham's F10 medium containing 10% fetal calf serum; for receptor activation experiments the medium was changed to 0.5% fetal calf serum 12 hours prior to stimulation.

### Example 4

### The C-RET Chimeric Receptor Promotes Differentiation and Survival of PC12 Cells

The biological activities of the chimeric receptor constructs were tested after it was determined that these constructs were expressed and active when stimulated with the CTA antibody, as shown in example 3. As a first test of the biological activities of these chimeric constructs, their effects on the differentiation and serum-free survival of the pheochromocytoma cell line PC12 were tested.

Lysates from PC12 cultures prepared two days after infection with viruses were probed for receptor expression with the CTA antibody. Strong expression of the full length and chimeric TRKA and MCK-10 receptors was observed. Lower levels of the C-RET and SEK receptors were expressed. The CTA antibody did not react with uninfected cultures or cultures infected with the β-galactosidase virus, as expected.

To examine the effects of these receptors on cell differentiation, the PC12 cells were first treated with virus overnight, and then replated under conditions which promote cellular differentiation. The cultures were incubated either in the presence of CTA IgG, to activate the introduced receptors, or NGF, to activate both endogenous and exogenous pools. The CTA antibody preparation could promote extensive outgrowth of the PC12 cells infected with the c-ret chimeric virus as well as with the full length and chimeric trkA viruses. The antibody had no obvious effect on RSV sek or RSV mck-10.

The percentage of cells undergoing differentiation was quantitated in each condition. RSV c-ret was able to promote differentiation of PC12 cells to a level comparable to that seen with either trkA construct. RSV c-ret virus showed a much higher activity in the absence of ligand than the trkA viruses did, most likely due to its auto-activation. No detectable activity of the sek or mck-10 constructs were observed.

The effects of the viruses on PC12 cell death was also tested. PC12 cells undergo programmed cell death in the absence of serum, and can be rescued by NGF. PC12 cells infected with the recombinant adenoviruses were tested for the ability of CTA antibody to rescue these cells from serum deprivation. The cells were infected as described above, and plated with and without CTA Antibody in serum free medium. MTT was used to detect surviving cells in the cultures two days later. The strongest effect on cell survival was observed for the two trkA receptor constructs. The C-RET chimeric receptor could prevent cell death as well, but its effect reached a plateau at a level below that of the trkA viruses. The RSV sek and RSV mck-10 viruses had marginal if any effect on the rescue of cells from death.

Hence, the C-RET cytoplasmic domain had biological activities comparable to those of TRKA in promoting PC12 cell differentiation and PC12 cell survival, while these activities were not detected after transfection with the sek or mck-10 receptor chimeras.

### Example 5

### Activation of the C-RET Chimera Promotes Survival of Embryonic Sympathetic Neurons

C-RET was activated in a ligand independent manner and tested for its ability to prevent apoptosis in embryonic sympathetic neurons. Sympathetic neurons extend processes and maintain viability and electrical excitability for weeks when cultured in the presence of NGF. In the absence of NGF, the neurons lose their processes, shrivel and undergo a classical program of apoptosis within hours. Activation of the TRKA receptor can stave apoptosis in sympathetic neurons without the addition of NGF.

The expression of chimeric receptor constructs was examined in rat sympathetic neurons by infecting the neurons, culturing them in the presence of RTA antibody, and lysing them 48 hours later. The RTA antibody was used to maintain their viability in the event that expression of a TRKA chimera would inhibit the function of the endogenous rat TRKA when NGF was used as a ligand. The lysates were immunoblotted and probed with the CTA antibody. Strong expression of RSV KP (TRKA chimera), RSV C-RET, and RSV SEK was observed. RSV MCK-10 showed somewhat weaker expression, especially of the 160 kDa isoform.

The neurons were transfected and plated in order to examine the effects of the chimeric receptors on neuronal cell survival. The infected neurons were cultured in the presence of either RTA antibody to activate their endogenous TRKA receptor, CTA antibody to activate the introduced receptor, or no addition to examine the background of cell survival. After three days, the cultures were stained with the vital dye calcein AM, and the number of surviving neurons in each condition was determined.

Uninfected sympathetic neurons responded well to the RTA antibody preparation, while the CTA antibody was ineffective in promoting cell survival. RTA could also sustain the survival of neurons infected with the RSV chimeric trkA, RSV c-ret, RSV sek, or RSV MCK-10 viruses. Moreover, infection of the neurons with the chimeric trkA virus resulted in the ability of the neurons to respond to CTA, and the level of survival measured in the presence of CTA reached levels equal to the survival promoted by RTA antibody. This indicated that survival of primary neurons could be promoted by introduction and activation of RPTKs.

Increased survival of sympathetic neurons was also observed when they were infected with the RSV c-ret virus and cultured with the activating CTA antibody. The level of survival reached approximately 50% of the maximal level attained with the RSV chimeric trkA virus. In addition, a substantial increase in survival in the absence of the activating antibody was measured. About 30% of the neurons survived compared to trkA chimera plus the activating antibody. This result was not unexpected, given the relatively high level of autoactivation detected with this chimeric receptor. Even though there was significant survival in the trkA chimera wells in the absence of CTA antibody, it was not as striking as with RSV c-ret. Finally, neither the RSV sek nor the RSV mck-10 virus could increase the survival of the neurons in the presence of CTA antibody, in contrast to the survival seen with RSV chimeric trkA and RSV c-ret.

The cells surviving through infection with the RSV c-ret virus were tested for the proper phenotype of a superior cervical ganglion neuron. Neurons were cultured with or without NGF for two days following infection with either the RSV c-ret or the trkA virus. The cultures were then subjected to immunofluorescence. In all cases neurons which were strongly expressing the chimeric receptors exhibited a healthy phenotype. When the cultures were supplemented with NGF, the cells expressing the transgene comprised a subset of surviving neurons, while in the absence of NGF, all large healthy neurons were found to be expressing the chimeric receptor. In addition, the neurons expressed comparable levels of tyrosine hydroxylase as compared to chimeric trkA controls. In cultures sustained by infection with the RSV c-ret virus only, the levels of tyrosine hydroxylase appeared to be highest in the cells expressing the highest levels of chimeric receptor, indicating that C-RET may be acting like the TRKA receptor in upregulating tyrosine hydroxylase in activity.

### Example 6

### Prevention of Sensory Neuron Programmed Cell Death by the C-RET Intracellular Region

C-ret constructs were transfected into DRG embryonic rat sensory neurons to determine whether the RPTK could promote survival in these cells as well as the sympathetic neurons studied in example 5.

All three of the receptors were robustly expressed in the DRG neuronal cell cultures. After plating the cells, both the TRKA and C-RET chimeric receptors could promote a significant level of neuronal survival, while the sek receptor construct was inactive. The level of survival promoted by c-ret in the presence of the CTA antibody was about 50% of that promoted by RSV trkA, and again there was significant survival in the absence of the antibody (31% of that measured with RSV trkA plus CTA antibody). Therefore, the survival activity of the c-ret receptor intracellular region extends to at least two major cell types, sympathetic and sensory neurons, in the peripheral nervous system.

### Example 7

### TRKA and C-RET Chimeric Receptors Promote MAPK Phosphorylation in Primary Neurons

The molecular mechanisms behind cell survival were explored once it was observed that activation of the c-ret chimera stimulated cell survival, as demonstrated in examples 5 and 6. In particular, the relationship between the c-ret intracellular region and the proteins that it potentially phosphorylated were explored.

Analysis of downstream signal transduction effectors of the TRKA and C-RET receptors showed significant similarities in certain effector systems. For example, GRB2 has been shown to bind one isoform of the C-RET receptor at its cytoplasmic tail; this interaction is predicted to activate the RAS/RAF/MAPK pathway. TRKA activates this pathway through interaction of the SHC adaptor protein with a phosphotyrosine located in the juxtamembrane region. Experiments were performed which determined that survival-promoting receptors could activate the MAPK pathway in sympathetic neurons.

Neurons were infected with virus constructs carrying either active or inactive trkA receptor genes or the chimeric c-ret receptor gene. The neurons were cultured in the presence of the RTA antibody for two days, and then starved for 3 hours. Half of the wells were stimulated for ten minutes with the CTA antibody, the cultures were lysed, and the resulting lysates immunoblotted. The immunoblots were probed with antibodies recognizing MAPK p42 and p44 (ERK1 and ERK2) or probed with antibodies recognizing only the phosphorylated form of MAPK.

CTA stimulation of the RSV chimeric trkA and RSV c-ret receptors efficiently and equivalently promoted MAPK phosphorylation. In addition, significant phosphorylation of MAPK with RSV c-ret was observed in the absence of stimulation, consistent with the observation that this construct was autoactivated. Thus, the correlation between the roles of trkA and c-ret in promoting primary neuronal survival and activating the MAPK pathway suggest that the MAPK pathway is a component of the apoptosis mechanism.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed.

In particular, although some formulations described herein have been identified by the excipients added to the formulations, the invention is meant to also cover the final formulation formed by the combination of these excipients. Specifically, the invention includes formulations in which one to all of the added excipients undergo a reaction during formulation and are no longer present in the final formulation, or are present in modified forms.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are within the following claims.

What is claimed is:

1. A method of evaluating a function of a receptor protein tyrosine kinase comprising the following steps:
(a) transfecting a nucleic acid vector into cells, wherein said vector encodes a chimera comprising an extracellular region and an intracellular region, wherein said intracellular region is from said receptor protein tyrosine kinase;
(b) contacting said cells with an antibody, wherein said antibody has specific binding affinity to said extracellular region; and
(c) monitoring an effect on said cells.

2. The method of claim 1, where said extracellular region consists of an extracellular region of a receptor protein tyrosine kinase selected from the group consisting of TRK, EGFR, PDGFR, and RET.

3. The method of claim 1, wherein said intracellular region consists of an intracellular region of an orphan receptor protein tyrosine kinase.

4. The method of claim 3, wherein said orphan receptor protein tyrosine kinase is selected from the group consisting of C-RET, SEK, MCK-10, AXL, TYRO3, MER, EPH, ECK, EEK, ERK, ELK, EHK1, EHK2, SEK, HEK, HEK2, MYK1, CEK9, MYK2, MDK1, IRR, CCK4, RYK, DDR, TYRO10, ROS, LTK, ALK, ROR1, ROR2, and TOR.

5. The method of claim 4, wherein said orphan receptor protein tyrosine kinase is C-RET.

6. The method of claim 1, wherein said cells and said extracellular region are from different species.

7. The method of claim 6, wherein said cells are mammalian.

8. The method of claim 6, wherein said extracellular region is isolated from a chicken.

9. The method of claim 1, wherein said antibody has specific binding affinity to a TRK extracellular region isolated from a chicken.

10. The method of claim 1, wherein said effect is a change or an absence of a change in cell phenotype.

11. A method of identifying compounds that modulate the function of a receptor protein tyrosine kinase in cells, wherein said method comprises the following steps:
(a) transfecting a nucleic acid vector into said cells, wherein said vector encodes a chimera comprising an extracellular region and an intracellular region, wherein said intracellular region is from said receptor protein tyrosine kinase;
(b) contacting said cells with one or more compounds;
(c) contacting said cells with an antibody, wherein said antibody has specific binding affinity to said extracellular region; and
(d) monitoring an effect on said cells.

12. The method of claim 11, wherein said extracellular region consists of an extracellular region of a receptor protein tyrosine kinase selected from the group consisting of TRK, EGFR, PDGFR, and RET.

13. The method of claim 11, wherein said intracellular region consists of intracellular region of an orphan receptor protein tyrosine kinase.

14. The method of claim 13, wherein said orphan receptor protein tyrosine kinase is selected from the group consisting of C-RET, SEK, MCK-10, AXL, TYRO3, MER, EPH, ECK, EEK, ERK, ELK, EHK1, EHK2, SEK, HEK, HEK2, MYK1, CEK9, MYK2, MDK1, IRR, CCK4, RYK, DDR, TYRO10, ROS, LTK, ALK, ROR1, ROR2, and TOR.

15. The method of claim 14, wherein said orphan receptor protein tyrosine kinase is C-RET.

16. The method of claim 11, wherein said cells and said extracellular region are from different species.

17. The method of claim 16, wherein said cells are mammalian.

18. The method of claim 16, wherein said extracellular region is isolated from a chicken.

19. The method of claim 11, wherein said antibody has specific binding affinity to a TRK extracellular region isolated from a chicken.

20. The method of claim 11, wherein said effect is a change or an absence of a change in cell phenotype.

21. The method of claim 11, wherein said effect is a change or an absence of a change in the catalytic activity of said intracellular region.

22. The method of claim 11, wherein said effect is a change or an absence of a change in an interaction between said intracellular region and a natural binding partner.

23. A method of identifying compounds that modulate the function of C-RET receptor protein tyrosine kinase comprising the following steps:
(a) expressing said C-RET in cells;
(b) contacting said cells with one or more compounds; and
(c) monitoring an effect on said cells.

24. The method of claim 23, wherein said effect is a change or an absence of a change in cell phenotype.

25. The method of claim 23, wherein said effect is a change or an absence of a change in catalytic activity of said C-RET receptor.

26. The method of claim 23, wherein said effect is a change or an absence of a change in the interaction between said C-RET receptor and a natural binding partner.
